(19) United States
(12) Patent Application Publication
(10) Pub. No.: US 2016/0279434 A1
(43) Pub. Date: Sep. 29, 2016

(54) A DEVICE, SYSTEM, METHOD, COMPUTER PROGRAM AND DATA SIGNAL FOR THE CONTROL OF A TRANSDERMAL DELIVERY DEVICE

(71) Applicant: INTERNATIONAL SCIENTIFIC PTY LTD, Leederville, WA (AU)

(72) Inventor: Jeffrey D EDWARDS, Leederville (AU)

(73) Assignee: INTERNATIONAL SCIENTIFIC PTY LTD, Leederville, WA (AU)

(21) Appl. No.: 14/442,287

(22) PCT Filed: Nov. 13, 2013
(86) PCT No.: PCT/AU2013/001311

§ 371 Date: May 12, 2015

(30) Foreign Application Priority Data
Nov. 14, 2012 (AU) 2012905028

(51) Int. Cl. A61N 2/00; A61N 1/30

(52) U.S. Cl. CPC A61N 2/002; A61N 1/30

(57) ABSTRACT

A method for programming a transdermal delivery device arranged to deliver a composition to a user, comprising the steps of receiving at least one variable parameter regarding a transdermal delivery method, utilising an algorithm to derive at least one control instruction for a transdermal delivery device utilising the at least one variable parameter, and providing the at least one instruction to the transdermal delivery device to effect the transdermal delivery method.

### TECHNICAL FIELD

The present invention relates to a device, system, method, computer program and data signal for the control of a transdermal delivery device. Embodiments of the invention find specific, but not exclusive, use in the control of a transdermal delivery device that utilises electromagnetic energy to vary the cellular environment of a membrane to facilitate transport of molecules across the membrane.

### BACKGROUND ART

The following discussion of the background art is intended to facilitate an understanding of the present invention only. The discussion is not an acknowledgement or admission that any of the material referred to is or was part of the common general knowledge as at the priority date of the application.

The delivery of active agents to or into membranes, such as skin and other biological components of the body must occur in sufficient amounts to allow the agent to achieve its purpose. Techniques and devices have been developed to assist in the transmission of active compositions through membranes, such as the skin. For example, International Patent Application No. PCT/AU2010/000782, filed by International Scientific Pty Ltd and entitled “An Apparatus and Method of Treatment Utilizing a Varying Electromagnetic Energisation Profile” (which is incorporated herein by reference) describes an apparatus which is designed to improve the delivery of desirable substances across a membrane by utilising a varying electromagnetic energisation profile.

The apparatus provides a delivery system that is of general applicability that can be used by a variety of individuals for a variety of needs.

However, the use of delivery systems which have general applicability results in a compromised efficacy when utilised on certain specific individuals and in certain specific environments. Different types of skin, different types of medical and genetic conditions, the nature of the composition and the ambient environment all have an effect on the efficacy of the delivery system on a given individual. As such, systems which have general applicability across a broad population and environment may be sub-optimal in their ability to deliver the most efficient and effective dose in very specific circumstances.

It is against this background that embodiments of the present invention have been developed.

### SUMMARY OF INVENTION

In a first aspect, the present invention provides a method for programming a transdermal delivery device arranged to deliver a composition to a user, comprising the steps of:

receiving at least one variable parameter regarding a transdermal delivery method;

utilising an algorithm to derive at least one control instruction for a transdermal delivery device utilising the at least one variable parameter; and

providing the at least one instruction to the transdermal delivery device to effect the transdermal delivery method.

In a second aspect, the present invention provides a system for programming a transdermal delivery device arranged to deliver a composition to a user, comprising:

a data input module arranged to receive at least one variable parameter regarding a transdermal delivery method;

a processor arranged to utilise an algorithm to derive at least one control instruction for a transdermal delivery device by processing the at least one variable parameter; and

an output module arranged to provide the at least one instruction to the transdermal delivery device to effect the transdermal delivery method.

In a third aspect, the present invention provides a transdermal delivery device arranged to deliver a composition to a user, comprising:

a data input module arranged to receive at least one variable parameter regarding a transdermal delivery method;

a processor arranged to utilise an algorithm to derive at least one control instruction for a transdermal delivery device by processing the at least one variable parameter; and

a transdermal delivery module arranged to utilise the at least one instruction to effect the transdermal delivery method.

In a fourth aspect, the present invention provides a computer program including at least one command, which, when executed on a computing system, is arranged to perform the method steps in accordance with the first aspect of the invention.

In a fifth aspect, the present invention provides a computer readable medium incorporating a computer program in accordance with the fourth aspect of the invention.

In a sixth aspect, the present invention provides a data signal encoding at least one command and being arranged to be receivable by at least one computing device, wherein, when the encoded command is executed on the computing system, the computing system performs the method steps in accordance with the first aspect of the invention.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is an example computing system which is capable of operating a device, system, method and/or computer program in accordance with an embodiment of the present invention;

FIGS. 2 to 6 are screenshots of a software program in accordance with an embodiment of the present invention; and

FIG. 7 is a flowchart depicting a process flow in accordance with an embodiment of the invention.

### DESCRIPTION OF EMBODIMENTS

### General Overview

The present invention relates generally to a device, system, method, computer program and data signal for the control of a transdermal delivery device.

In more detail, one aspect of the embodiments described herein provides a method for programming a transdermal delivery device arranged to deliver a composition to a user, comprising the steps of receiving at least one variable parameter regarding a transdermal delivery method, utilising an algorithm to derive at least one control instruction for a transdermal delivery device utilising the at least one variable parameter, and providing the at least one instruction to the transdermal delivery device to effect the transdermal delivery method.

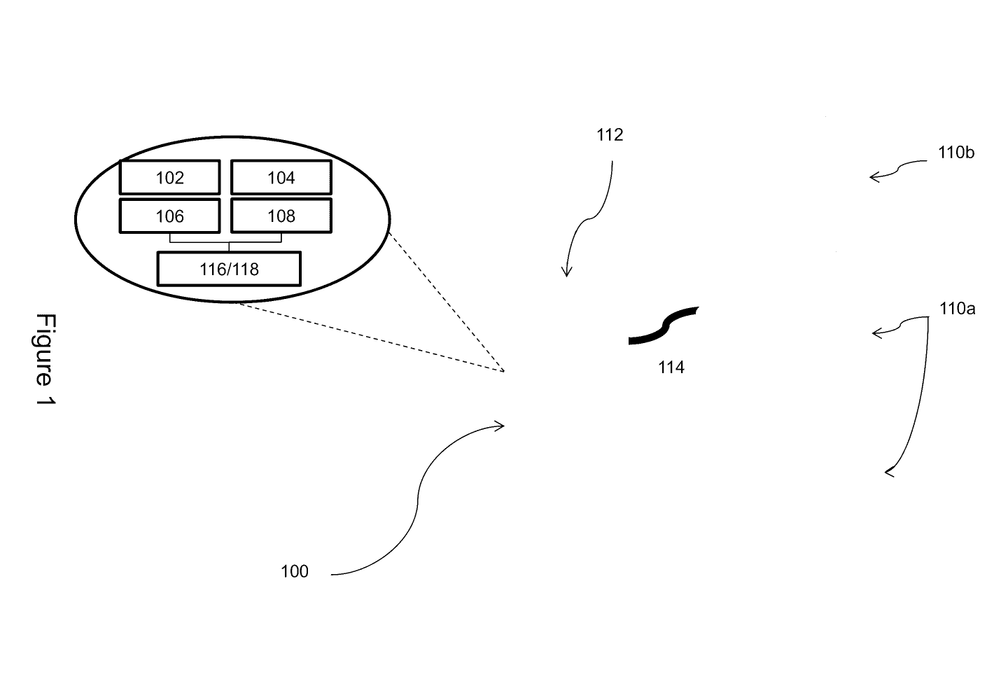

The method is codified in a computing system, such as the computing system shown at FIG. 1.

In FIG. 1 there is shown a schematic diagram of a computing system, which in this embodiment is a computing system 100 suitable for use with an embodiment of the present invention. The computing system 100 may be used to execute application and/or system services such as a system and method for facilitating the controlling, monitoring and issuing of commands in accordance with an embodiment of the present invention.

With reference to FIG. 1, the computing system 100 may comprise suitable components necessary to receive, store and execute appropriate computer instructions. The components may include a processor 102, read only memory (ROM) 104, random access memory (RAM) 106, an input/output devices such as disc drives 108, remote or connected input devices 110 (such as a mobile computing device, a smartphone or a ‘desktop’ personal computer), and one or more communications link(s) 114.

The computing system 100 includes instructions that may be installed in ROM 104, RAM 106 or disc drives 112 and may be executed by the processor 102. There may be provided a plurality of communication links 114 which may variously connect to one or more computing devices 110a such as servers, personal computers, terminals, wireless or handheld computing devices, or mobile communication devices such as a mobile (cell) telephone. The communications links may also connect to one or more transdermal delivery devices 110b. At least one of a plurality of communications link 114 may be connected to an external computing network through a telecommunications network.

In one particular embodiment the device may include a database 116 which may reside on the storage device 112. It will be understood that the database may reside on any suitable storage device, which may encompass solid state drives, hard disc drives, optical drives or magnetic tape drives. The database 116 may reside on a single physical storage device or may be spread across multiple storage devices.

The server 100 includes a suitable operating system 118 which may also reside on a storage device or in the ROM of the server 100. The operating system is arranged to interact with the database and with one or more computer programs to cause the server to carry out the steps, functions and/or procedures in accordance with the embodiments of the invention described herein.

Broadly, the invention relates to a computing method and system arranged to interact with one or more transdermal delivery devices via a communications network. The transdermal delivery devices 110b may be physically distinct (separate) from the computing devices 110a as shown in FIG. 1, but the transdermal computing device may also include an integrated computing device capable of functioning in the same manner as computing devices 110a. This embodiment will be described in more detail later.

The system, in one embodiment, utilises a server including the database 116 to contain information pertaining to permeability profiles. The database 116 is arranged to receive the information via the communications network from the one or more remote devices 110a and to subsequently communicate information to one or more transdermal delivery devices 110b.

Other aspects of the broad inventive concept relate to a corresponding device, method, computer program, computer readable media and data signal. The method facilitates the transfer of information to and from the transdermal delivery devices, via the computing system. The centralised database receives a request to provide information to the one or more transdermal delivery devices, and forwards the information via a communications network to the one or more remote transdermal delivery devices.

### Transdermal Delivery Device

In the context of the broader invention described herein, the transdermal delivery device used as an illustrative example is the transdermal delivery device described in more detail in International Patent Application No. PCT/AU2010/000782. It will be understood, however, that the broader invention described herein is applicable to and finds use with any type or form of transdermal delivery device, including but not limited to an electromagnetophoresis device, sonophoresis device, iontophoresis device, thermophoresis device, micro-current device or low-level laser therapy device.

For a further example of a powered means for driving a transdermal delivery device, the electromagnetophoresis device of International Patent Application No. PCT/AU2004/001599 may be used to enhance penetration of the active agents into the skin. Other examples of powered devices include US Patent Application No. 2002/0147424.

The transdermal delivery device of the present example utilises a diffusion enhancement technique that increases the rate at which the active agent is delivered from the device for delivering an active agent to a dermal or mucosal surface, to the dermal or mucosal surface itself. This involves, at least in part, the use of a technique for altering the permeability of the dermal or mucosal surface to increase the amount of active agent which actually penetrates the dermal or mucosal surface.

The permeability of the dermal or mucosal surface is varied by use of an electromagnetic field generating device including a coil which has electrical connectivity at both ends to allow unidirectional flow of current.

In turn, the coil is operated via a control device arranged to control the field generating device to alternately produce active and substantially inactive electromagnetic field portions.

In the example device, each active electromagnetic field portion has a frequency of between 10 Hz and 100 Hz at a field strength of between 1 and 50 Gauss and a plurality of generally rectangular electromagnetic field pulses wherein each electromagnetic field pulse has a duration of between 25 μs and 100 ms.

The control device further includes a microcontroller, which may be programmable by a user via the computing device 110a (or 100) of FIG. 1, so that an electromagnetic signal corresponding to a predetermined therapeutic substance delivery plan is produced.

The microcontroller may be programmed such that dermal permeability is increased at one or more specific times, permeability is increased for a specific period of time, and so on. For example, the microcontroller may be configured so that the apparatus carries out a specific treatment plan, by generating an appropriate energisation signal pattern and using the energisation signal to apply one or more specific electromagnetic field patterns to a target area of a patient at specific times, for a specific time duration and/or in response to the specific characteristics of the active agent being delivered.

In the context of the example described herein, the transdermal delivery device preferably produces and delivers an electromagnetic field to the dermal or mucosal surface wherein the field delivered is defined by a mnemonic profile of:

[(A-B-C-D)E], [(A1-B1-C1-D1)E1]

where:

A and A1 respectively define the number of 400 μs time units that the electromagnetic field pulse is on for wherein each of A and A1 is a number between 0.1 and 10;

B and B1 respectively define is the number of 400 μs time units the field is off for wherein B is a number between 0.1 and 100, while B1 is a number between 0.1 and 100;

C is a number between 1 and 255, which defines the number of times the A and B combination is repeated, while C1 is also a number between 1 and 255 and defines the number of times the A1 and B1 combination is repeated;

D and D1 respectively define the number of 400 μs time units that the field is off for wherein D is a number between 0 and 255, while D1 is a number between 0 and 255; and

E defines the number of times the A to D envelope is executed before moving onto the [(A1-B1-C1-D1)E1] packet, while E1 defines the number of times the A1 to D1 envelope is executed before moving onto the next mnemonic profile wherein E and E1 are respectively numbers between 1 and 25.

The mnemonic profile selected can differ according the drug administered and also due to a number of environmental conditions, including but not limited to the skin type of a user, the general environment (i.e. the local temperature, humidity and pressure) and any other factors which may affect the efficacy of the transdermal delivery.

In use, a mnemonic profile type is selected by the device as disclosed later in this document. In particular, the mnemonic profile type is selected according to the nature and properties of the formulation or active ingredient being administered.

By way of example, a mnemonic of [(2-12-100)255], [(2-12-50)155] (i.e. A=2, B=12, C=100, D=0, E=255, A1=2, B1=12, C1=50, D1=0, E1=155) is one possible mnemonic that has been shown through research and testing to provide enhanced delivery of a water soluble molecule of around 400 daltons from a conventional oil in water emulsion when applied to a person of Caucasian skin type, between 50-60 years of age in standard temperate and atmospheric pressure conditions.

However, individuals who have different skin characteristics may not be well served by this standard mnemonic, in which case, the mnemonic is adjusted depending on their individual skin characteristics.

By way of example only, if the person was of Chinese ancestry and 25 years of age, the mnemonic would optimally need to be adjusted to accommodate the higher skin lipid content and the less effective hydrophilic delivery pathway that would otherwise be available in Caucasian skin types. In such a case, the mnemonic may be optimised to program the transdermal delivery device with a mnemonic of [(2-12-75)255], [(2-18-100)100] which changes the lipophilicity of the mnemonic and provide greater diamagnetic force to overcome the higher thermodynamic barrier to partitioning of the active ingredient into a more oily environments.

In yet another example, the same formulation may be optimised to suit an older person of Caucasian ancestry living in a hot and dry climate. In such a case, natural perspiration and the underlying skin type may provide an efficient hydrophilic path via the skin's appendageal system that can be further enhanced by modifying the mnemonic to [(4-12-50)255], [(2-12-50)255]. In such a case the greater period of magnetic flux may increase diamagnetic repulsion, while the more inductive portion of the second part of the mnemonic may enhance partitioning of the active ingredient into this more aqueous environment.

In yet another example, a similar older person of Caucasian ancestry with physical skin characteristics (as described in the previous paragraph) but living in a hot and dry climate may wish to apply an anti-aging formulation made up of a high molecular weight oligopeptide of 800 daltons in size in a lipophilic formulation. In such case, natural perspiration and underlying skin lipid content may provide a challenging delivery pathway. Aided by the data relating to both the user and the nature of the formulation, a modified mnemonic of [(5-25-50)10], [(2-50-2)50] may be used to overcome the thermodynamic barriers to partitioning and diffusion into the skin and enhanced delivery via the lipid bilayer's storage potentials.

More generally, the inventor of the embodiments and the broader inventive concept described and defined herein, has discovered that age may have a significant effect on skin lipid density, which in turn alters the efficiency of the lipophilic delivery pathway. The embodiments described herein provide a system and method of adjusting the delivery fields to accommodate such personal characteristics. Furthermore, the presence of any dermatological condition such as eczema or psoriasis can significantly alter dermal barrier function, and through the system and method is provided to adjust any delivery or treatment to an individual's characteristics.

In this manner, information provided by the user regarding their individual skin types and characteristics may be used to alter or modify a mnemonic suitable for a specific formulation or active ingredient resulting in an optimised delivery of said formulation or active ingredient for that individual.

### Example of Programming of Mnemonic Profile via User Interface

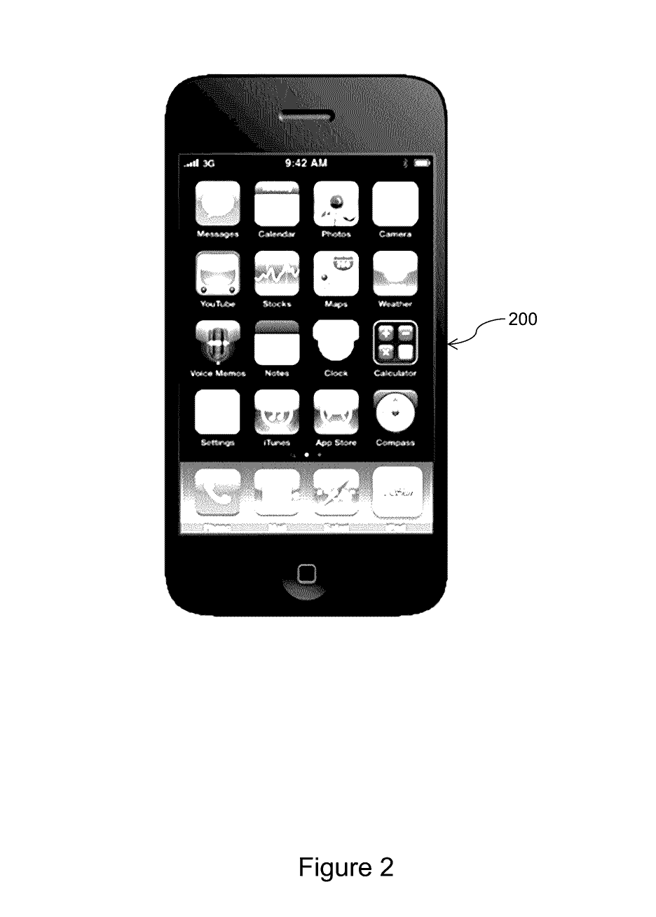

The embodiment is hereinbelow described with reference to a simple example and with reference to FIGS. 2 to 7 and the preceding description. FIGS. 2 to 6 illustrate a user interface provided by a software program (‘app’) in accordance with an embodiment of the invention.

An icon for the app is illustrated generally at 200 in FIG. 2. To access the app, a user ‘touches’ the icon in accordance with the general convention for operating applications on a ‘smartphone’.

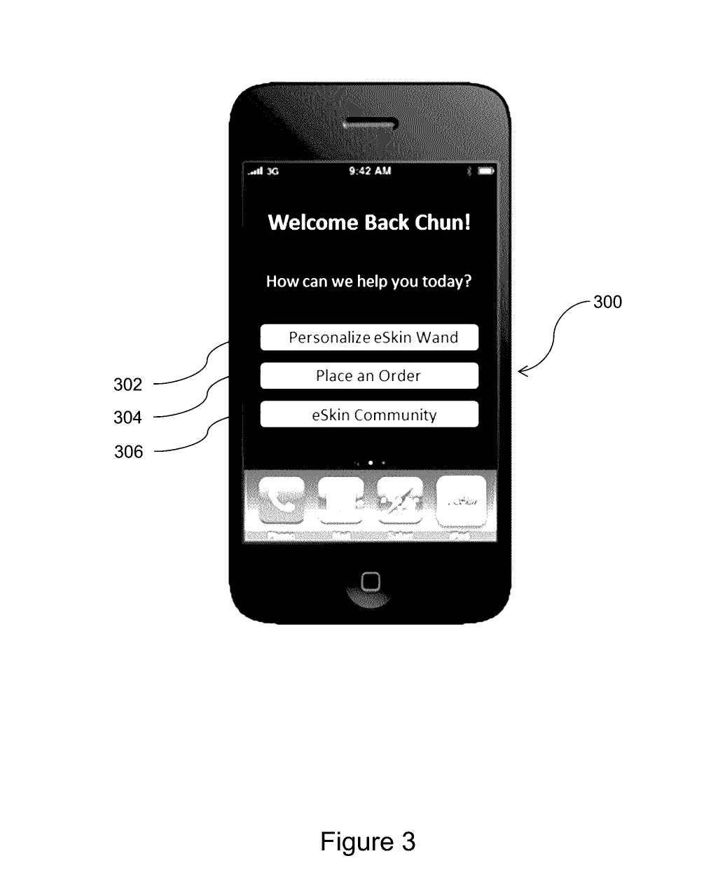

When a user touches the icon, the application is loaded into the processor and memory of the device and the user is presented with a ‘welcome screen’ 300 (shown in FIG. 3). The welcome screen 300 provides three options to the user. The user may personalise the transdermal delivery device (icon 302), place an order for further products (icon 304) or interact with a community of other users (icon 306).

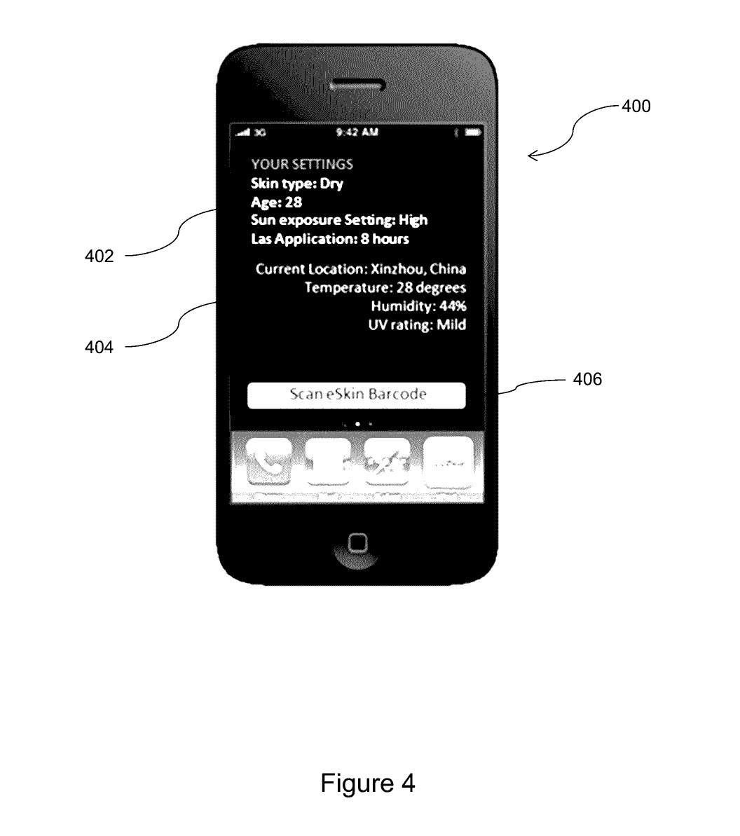

The following description describes, in detail, the process of personalising the transdermal delivery device. Referring to FIG. 4, there is shown a user interface 400 that allows a user to provide specific personal and environmental information.

At area 402, there is provided a number of fields (which are linked to parameters) which relate to personal factors that have an effect on the mnemonic profile of the field that is to be utilised by the transdermal delivery device.

In the example provided, the parameters include the skin type of the user, the age of the user, the sun exposure of the user and the last time the user applied the composition. However, it will be understood that other parameters such as ethnicity of the user, age, skin lipids, dryness, colouring and dermatological conditions may also be included, as required.

At area 404, there is provided a number of fields (which are linked to parameters) which relate to environmental factors that have an effect on the mnemonic profile of the field that is to be utilised by the transdermal delivery device.

In the example provided, the parameters include the current geographical location of the user, the current temperature, the current humidity and the current UV rating. However, it will be understood that other parameters may also be included such as the use of air-conditioning, time of activity and duration of activity. These parameters are then utilised to determine a suitable mnemonic, as described previously. The parameters may be determined in any suitable manner. For example, the app may include a “look up”table which maps a user's selection to relevant values for A, B, C, D, E, A1, B1, C1, D1 and E1 as required.

At area 406, there is provided an icon which allows the user to gather information regarding the nature of the composition that is to be administered to the user. On touching icon 406, the user then uses their smartphone to scan a barcode (or QR code) on packaging associated with the composition.

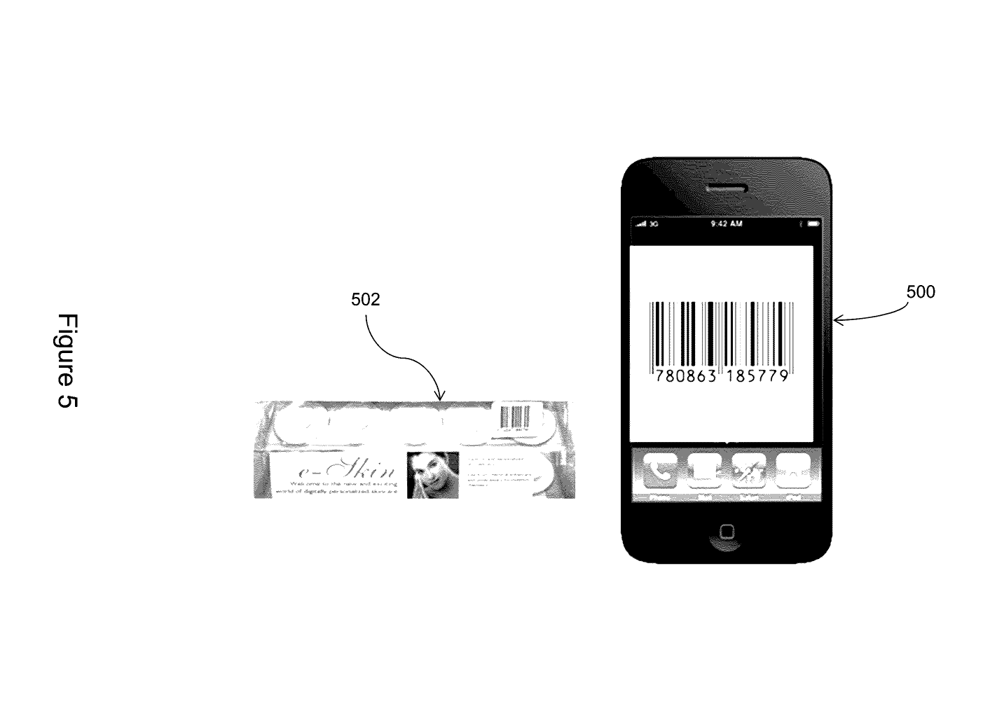

As shown in FIG. 5, the smartphone 500 scans the packaging 502 to obtain information regarding the composition. By scanning the barcode, the smartphone 500 receives information which can then be used to access a database (not shown, but equivalent to database 116 described previously), which contains more information about the product. In particular, the further information provides information that assists in the calculation of the most desirable mnemonic profile, as described above.

Once all information has been gathered, the smartphone 500 utilises an algorithm to calculate the most desirable mnemonic profile.

Such an algorithm as described previously, is a sequence of numerical values through which the standard delivery profile for a known formulation of active ingredient is altered to suit individual users.

By way of example only, a standard delivery field profile, developed by experimentation and research to enhance the transdermal delivery of a retinoid based anti-aging formulation may be [(1-14-100)125], [(1-14-50)155] (i.e. A=1, B=14, C=100, D=125, A1=1, B1=14, C1=50, D1=155).

By way of example, entering an age between 25-35 years may necessitate a reduction in C from 100 to 50, and C1 from 50 to 40 in order to accommodate for the greater skin lipids and higher vascularly of younger skin. In another example, the geographic location, provided either by the users or automatically collected by the smartphone network or communications device, may indicate the user is in the tropics. In such high temperature and relative humidity conditions, the more efficient delivery pathway will be the hydrophilic appendageal pathway. If the user further indicates that they are of Chinese ancestry, these two factors suggest that the profile should optimally altered by increasing E and E1 by 25 units each and decreasing B by 2 units and B1 by 4 units to accommodate these individual characteristics.

In this manner, a standard profile of [(1-14-100)125], [(1-14-50)155] may be adjusted to accommodate various individual parameters and result in a profile of (1-12-50)150], [(1-10-40)180] through use of the aforementioned interface.

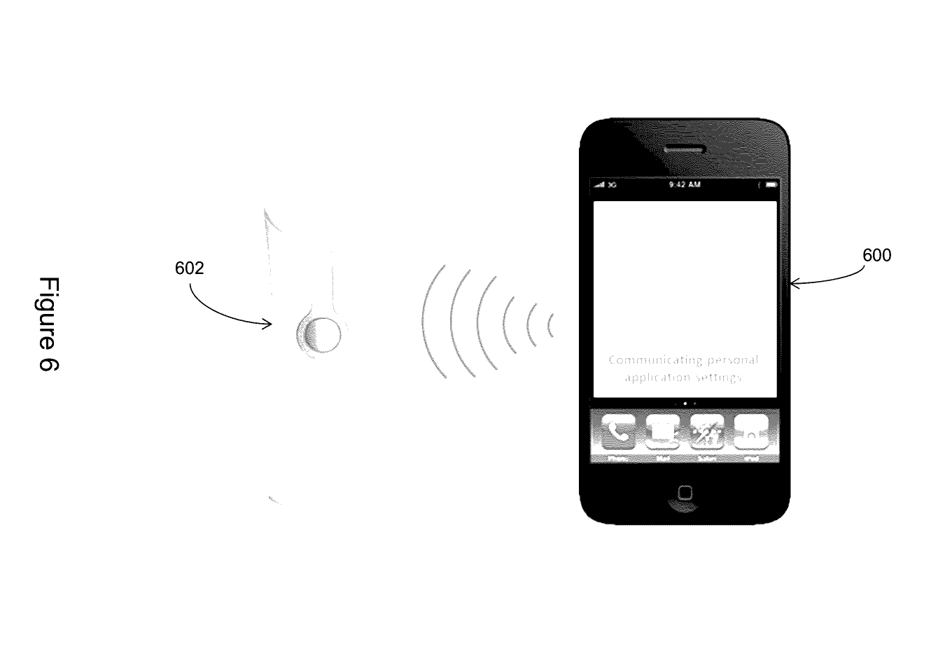

At FIG. 6, there is shown a smartphone 600 transmitting the instructions (including the desired mnemonic profile) to a transdermal delivery device 602. One the device 602 has received the instructions, a user may operate the device to deliver the composition.

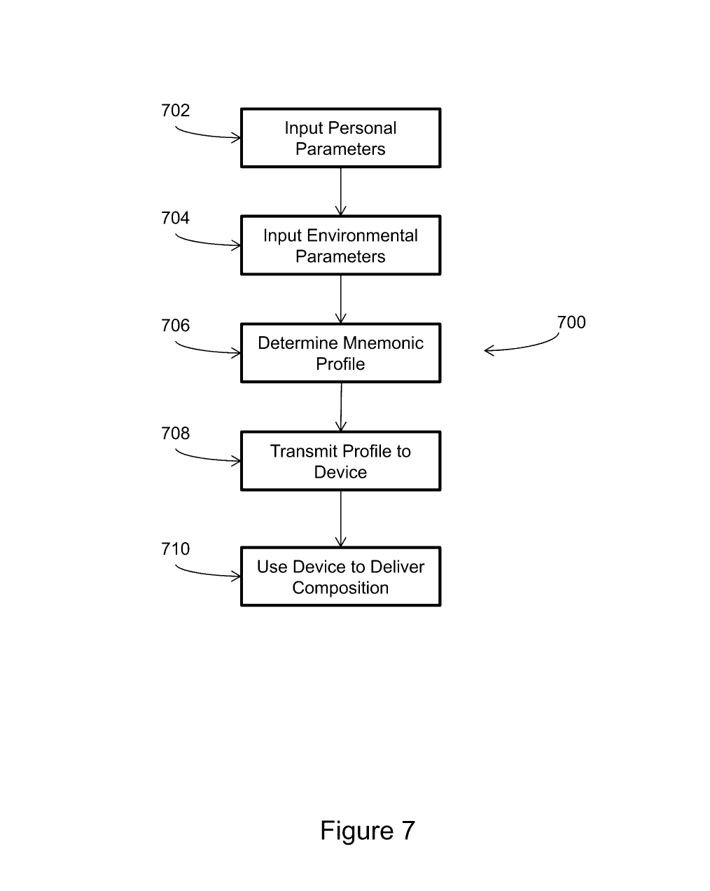

The process described above is summarised by the flowchart 700 of FIG. 7. At 702, the user inputs personal parameters. At step 704, the user inputs environmental parameters. At step 706, the information is used to determine a mnemonic profile. At step 708, the profile is subsequently transmitted to the device and at step 710, the device is used to deliver the composition.

The profile may be determined in the local application (i.e. in the smartphone app) which would reside on a device such as device 110a, or alternatively, the mnemonic profile may be determined on a computing system 100, such as the computing system 100 shown in FIG. 1.

That is, the embodiments described herein contemplate two different modes of determination. In some instances, the ‘app’ may be completely self-contained and may not require communication with an external computing system in order to carry out step 706. That is, a database may reside within the smartphone. In another embodiment, the ‘app’ may merely retrieve information and forward the information to the computing system 100, so that the computing system may calculate the mnemonic profile before providing the profile to the transdermal device.

Once the mnemonic profile has been determined, the profile is transmitted to the transdermal delivery device 110b. Where the mobile device 110a is integral with the transdermal delivery device 110b, it will be understood that the ‘transmission’ step may occur between different hardware or software modules within the same physical device.

In the embodiment shown with reference to FIG. 1, the transmission of the mnemonic profile may occur directly between device 110a and 110b, utilising a suitable protocol, such as 802.11 wireless transmission, Bluetooth, or any other suitable transmission format.

In another embodiment, also contemplated with respect to FIG. 1, the mnemonic profile may be transmitted from the computing system 100 to the transdermal delivery device 110b.

Other variations in transmission between the three devices, namely the computing system 100, the transdermal delivery device 110b and the mobile device 110a all fall within the purview of the broader inventive concept described herein, and the embodiments described above are to be taken as illustrative but not restrictive.

Once the mnemonic profile has been transferred to the transdermal delivery device 110b, the device may then be used to deliver the composition, in any suitable manner, such as the manner described more generally in International Patent Application No. PCT/AU2010/000782.

### Advantages

One of the advantages of the embodiments and broader invention described herein is that the invention removes the onus from consumers to correctly ‘set up’ or ‘optimise’ their transdermal delivery device. One common problem with devices that require customisation is that non-expert users frequently fail to correctly set-up and operate devices of moderate or high complexity. This results in sub-optimal results and general dissatisfaction with the device. Moreover, in certain limited examples, the incorrect use of the device could cause injury. As such, the provision of a ‘smart’ system ameliorates the possibility of user error and of subsequent dissatisfaction or user injury.

### Disclaimers

Throughout this specification, unless the context requires otherwise, the word “comprise” or variations such as “comprises” or “comprising”, will be understood to imply the inclusion of a stated inte.g.er or group of integers but not the exclusion of any other integer or group of integers.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Although not required, the embodiments described with reference to the method, computer program, data signal and aspects of the system can be implemented via an application programming interface (API), an application development kit (ADK) or as a series of program libraries, for use by a developer, for the creation of software applications which are to be used on any one or more computing platforms or devices, such as a terminal or personal computer operating system or a portable computing device, such as a smartphone or a tablet computing system, or within a larger server structure, such as a ‘data farm’ or within a larger transaction processing system.

Generally, as program modules include routines, programs, objects, components and data files that perform or assist in the performance of particular functions, it will be understood that the functionality of the software application may be distributed across a number of routines, programs, objects or components to achieve the same functionality as the embodiment and the broader invention claimed herein. Such variations and modifications are within the purview of those skilled in the art.

It will also be appreciated that where methods and systems of the present invention and/or embodiments are implemented by computing systems or partly implemented by computing systems then any appropriate computing system architecture may be utilised. This includes standalone computers, network computers and dedicated computing devices (such as field-programmable gate arrays).

Where the terms “computer”, “computing system” and “computing device” are used in the specification, these terms are intended to cover any appropriate arrangement of computer hardware for implementing the inventive concept and/or embodiments described herein.

Where reference is made to communication standards, methods and/or systems, devices may transmit and receive data via a variety of forms including but not limited to 3G, 4G (CDMA/GSM), Wi-Fi, Bluetooth, etc., and/or via any type of radio frequency, optical, acoustic, magnetic, or any other form of communication that may become available from time to time.

1. A method for programming a transdermal delivery device arranged to deliver a composition to a user, comprising the steps of:
receiving at least one variable parameter regarding a transdermal delivery method:
utilising an algorithm to derive at least one control instruction for a transdermal delivery device utilising the at least one variable parameter; and
providing the at least one instruction to the transdermal delivery device to effect the transdermal delivery method.

2. A method in accordance with claim 1, wherein the at least one variable parameter is at least one of an ambient temperature value, an ambient atmospheric pressure value, and an ambient humidity value.

3. A method in accordance with claim 1, wherein the at least one variable parameter is a characteristic of the composition of the substance to be delivered by the transdermal delivery device.

4. A method in accordance with claim 1, wherein the at least one variable parameter is a characteristic of the dermal layer to which the transdermal delivery device is to be applied.

5. A method in accordance with claim 1, wherein the at least one variable parameter is a geographical location of the user.

6. A method in accordance with claim 1, wherein the at least one instruction is provided utilising a wireless communication device.

7. A method in accordance with claim 1, wherein the transdermal delivery device is an electromanegtopeherisis device.

8. A system for programming a transdermal delivery device arranged to deliver a composition to a user, comprising:
a data input module arranged to receive at least one variable parameter regarding a transdermal delivery method;
a processor arranged to utilise an algorithm to derive at least one control instruction for a transdermal delivery device by processing the at least one variable parameter; and
an output module arranged to provide the at least one instruction to the transdermal delivery device to effect the transdermal delivery method.

9. A system in accordance with claim 8, wherein the at least one variable parameter is at least one of an ambient temperature value, an ambient atmospheric pressure value, and an ambient humidity value.

10. A system in accordance with claim 8, wherein the at least one variable parameter is a characteristic of the composition of the substance to be delivered by the transdermal delivery device.

11. A system in accordance with claim 8, wherein the at least one variable parameter is a characteristic of the dermal layer to which the transdermal delivery device is to be applied.

12. A system in accordance with claim 8, wherein the at least one variable parameter is a geographical location of the user.

13. A system in accordance with claim 9, wherein the at least one instruction is provided utilising a wireless communication device.

14. A system in accordance with claim 8, wherein the transdermal delivery device is an electromagnetophoresis device.

15. A transdermal delivery device arranged to deliver a composition to a user, comprising:
a data input module arranged to receive at least one variable parameter regarding a transdermal delivery method;
a processor arranged to utilise an algorithm to derive at least one control instruction for a transdermal delivery device by processing the at least one variable parameter; and
a transdermal delivery module arranged to utilise the at least one instruction to effect the transdermal delivery method.

16. A computer program including at least one command, which, when executed on a computing system, is arranged to perform the method steps of claim 1.

17. A computer readable medium incorporating a computer program in accordance with claim 16.

18. A data signal encoding at least one command and being arranged to be receivable by at least one computing device, wherein, when the encoded command is executed on the computing system, the computing system performs the method steps of claim 1.
